(19) United States
(12) Patent Application Publication
(10) Pub. No.: US 2014/0309130 A1
(43) Pub. Date: Oct. 16, 2014

(54) Use of MicroRNAs for Screening and Diagnosis of Prostate Cancer and Benign Prostatic Hyperplasia

(71) Applicant: Taha Alexander Haj-Ahmad, St. Catharines (CA)

(72) Inventor: Taha Alexander Haj-Ahmad, St. Catharines (CA)

(73) Assignee: Norgen Biotek Corporation, Thorold (CA)

(21) Appl. No.: 14/219,165

(22) Filed: Mar. 19, 2014

Related U.S. Application Data
(60) Provisional application No. 61/812,035, filed on Apr. 15, 2013.

(51) Int. Cl. C12Q 1/68

(52) U.S. Cl. CPC C12Q 1/6886
USPC 506/9; 435/6.12; 435/6.11

(57) ABSTRACT

Disclosed are microRNA biomarkers and use thereof for screening and diagnosis of prostate cancer and benign prostatic hyperplasia. This invention provides a method for screening for prostate cancer in a subject involving the steps of: (a) assaying the miRNA expression level in a test sample from the subject to be screened for prostate cancer; (b) comparing the assayed miRNA expression level of the subject to the miRNA expression level in a normal sample providing a control relative to the test sample of the subject; and (c) computing the differential expression of the miRNA from the subject, wherein the over-expression of miR-1825 or under the expression of miR-484 is indicative of prostate cancer. In another aspect, provided is a method for screening for benign prostatic hyperplasia in a subject involving the steps of: (a) assaying the miRNA expression level in a test sample from the subject to be screened for benign prostatic hyperplasia; (b) comparing the assayed miRNA expression level of the subject to the miRNA expression level in a normal sample providing a control relative to the test sample of the subject; and (c) computing the differential expression of the miRNA from the subject, wherein the under expression of miR-498 is indicative of benign prostatic hyperplasia.

### RELATED PATENT APPLICATIONS

This application claims the benefit of U.S. provisional patent application Ser. No. 61/812,035, filed on Apr. 15, 2013 under U.S.C. §119(e) (hereby specifically and fully incorporated herein by the reference).

### REFERENCE TO SEQUENCE LISTING, A TABLE FOR A COMPUTER PROGRAM LISTING, COMPACT DISC APPENDIX

The contents of the text file named “Sequence Listing 140226_ST25.txt” (created Feb. 26, 2014 and being 1 KB in size), are hereby incorporated by reference in their entirety.

### FIELD OF INVENTION

The present invention relates to the use of microRNAs for screening and diagnosis of prostate cancer and benign prostatic hyperplasia.

### BACKGROUND

A number of techniques are currently available for assessing prostate health and for screening prostate cancer (PCa). The most common methods for screening PCa include digital rectal exam, trans-rectal ultrasonography, and prostate specific antigen-based testing (PSA) (Mettlin, et al., 1991; Catalona, et al., 1991).

The most commonly used method for screening PCa is PSA testing. Serum PSA testing for PCa is relatively inexpensive and has high sensitivity for PCa (86%) (Hoffman, et al., 2002). PSA testing unfortunately has a notoriously poor specificity (33%) for detecting PCa. This is because other conditions such as benign prostatic hyperplasia (BPH), prostatitis and age-related increases in PSA can also result in an elevated level of serum PSA, which is why PSA testing often leads to false positive results (Hoffman, et al., 2002). It has also been reported that PCa was detected in approximately 15% of men with normal PSA values (<4 ng/mL), making PCa screening a challenge by PSA alone. These findings have fueled the persistent conflict centered around the benefits of regular PSA screening, and have led many to question the value of PSA as an early detection tool (Carter and Isaacs, 2004; Croswell, et al., 2011; Prensner, at al., 2012). Criticisms such as these have been fueling the search for newer and better markers for PCa, including improvements to PSA itself (Madu and Lu, 2010).

Much research has therefore gone into discovering non-PSA markers for prostate cancer, and numerous potential candidates have been reported in the literature. Some of these candidates include prostatic acid phosphatase (PAP), α-Methylacyl Coenzyme-A Racemase (AMACR), Glutathione S-transferases (GSTs), Chromogranin-A (CgA), Prostate Specific Membrane Antigen, Sarcosine, Caveolin-1 (Cav-1), prostate cancer gene-3 (PCA3), Exosome-Based Markers, Disabled homolog-2 interacting protein (DAB2IP), Transmembrane serine protease (TMPRSS2) and Spond in-2.

Furthermore, there are many cases in the literature of microRNAs being used as biomarkers for diagnosing and monitoring various diseases, including cancer. MicroRNAs (miRNAs) are a class of short (˜22 nt), single stranded RNA molecules that function as post-transcriptional regulators of gene expression (Bartel, 2009). MicroRNAs can regulate a variety of important biological pathways, including: cellular proliferation, differentiation and apoptosis (He and Hannon, 2004). miRNAs have also been found to play a significant role in carcinogenesis (Cho, 2007). They do so via their ability to function as regulators of tumour suppressors and oncogenes (Esquela-Kerscher and Slack, 2006). As a result of their regulatory nature, patterns of miRNA expression appear to be tissue and even tumour specific (Lagos-Quintana et al., 2002). Moreover, profiling of miRNA expression patterns was shown to be more useful than the equivalent mRNA profiles for characterizing poorly differentiated tumours (Lu et al., 2005). As such, miRNA expression signatures are expected to offer serious potential for diagnosing and prognosing cancers of any provenance (Yanaihara, et al., 2006; Lu, et al., 2005).

In 2006 Volinia, et al. published an analysis of miRNA expression patterning that was derived from 56 prostate tumours as well as tissues from 7 healthy individuals—together they identified 39 miRNAs that were commonly up-regulated and 6 that were commonly down-regulated among the cancerous samples (Volinia, et al., 2006). Deregulation in PCa, of at least some of the same miRNAs was again observed, when in 2008 Ambs, et al. published their observations (also using tissue samples) (Ambs, et al., 2008). Together these studies would seem to suggest that the up-regulation of miRNAs: 32; 26a; 196a; 181a; 25; 93; 92; let-7i and the down-regulation of miRNAs: 218; and 128 might be a common occurrence in at least some prostate tumours. In another study, Porkka, et al. analyzed miRNA expression in 6 PCa cell lines, 9 PCa xenografts, 4 BPH tissue samples, 5 untreated prostatic carcinoma tissue samples and 4 hormone-refractory prostatic carcinoma samples. They also observed deregulation among a number of miRNAs, however with results that varied from the previously mentioned studies. While differences in methodology and sampling likely form the basis for such inconsistencies, it has through studies like these that an association between PCa and several miRNAs has already been tentatively established (Coppola, et al., 2010). To date however, no miRNA-based diagnostic test has yet been approved for the early detection of PCa.

### SUMMARY OF INVENTION

In one aspect, provided is a method for screening for prostate cancer in a subject. The steps of this method for screening for prostate cancer include: (a) assaying the miRNA expression level in a test sample from the subject to be screened for prostate cancer; (b) comparing the assayed miRNA expression level of the subject to the miRNA expression level in a normal sample providing a control relative to the test sample of the subject; and (c) computing the differential expression of the miRNA from the subject, wherein the over-expression of miR-1825 or the under expression of miR-484 is indicative of prostate cancer.

In another aspect of this invention, a method for screening for benign prostatic hyperplasia in a subject is provided. This method includes the steps of: (a) assaying the miRNA expression level in a test sample from the subject to be screened for benign prostatic hyperplasia; (b) comparing the assayed miRNA expression level of the subject to the miRNA expression level in a normal sample providing a control relative to the test sample of the subject; and (c) computing the differential expression of the miRNA from the subject, wherein the under expression of miR-498 is indicative of benign prostatic hyperplasia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates that expression of 18 significantly deregulated miRNAs between pooled PCa, BPH and Healthy Control as detected by microarray;

FIG. 2 is a bar graph illustrating the deregulation of miR-1825 and miR-484 among urine samples from the PCa group; and

FIG. 3 is a bar graph illustrating the down-regulation of miR-498 among urine samples from the BPH group and the PCa group.

### DESCRIPTION

The present invention may be understood more readily by reference to the following detailed description of the invention. It is to be understood that this invention is not limited to the specific devices, methods, conditions or parameters described herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

These and other aspects, features and advantages of the invention will be understood with reference to the detailed description herein, and will be realized by means of the various elements and combinations particularly pointed out in the appended claims. It is to be understood that both the foregoing general description and the following detailed description of the invention are exemplary and explanatory of preferred embodiments of the inventions, and are not restrictive of the invention as claimed. Unless defined otherwise, all technical and scientific terms used herein have the same meaning as commonly understood by one of ordinary skill in the art to which this invention belongs.

As described in the Background of the Invention, a number of problems are currently associated with PSA testing for prostate cancer. Chief among these is a high incidence of false positive results. These can result from other conditions such as benign prostatic hyperplasia and prostatitis that can also lead to an elevated level of serum PSA (Hoffman, 2002). Provided herein are non-invasive and non-PSA-based methods for screening and diagnosis of prostate cancer and benign prostatic hyperplasia. The disclosed methods employ prostate cancer and benign prostatic hyperplasia specific microRNA biomarkers.

In one aspect, provided is a method for screening for prostate cancer in a subject comprising the steps of: (a) assaying the miRNA expression level in a test sample from the subject to be screened for prostate cancer; (b) comparing the assayed miRNA expression level of the subject to the miRNA expression level in a normal sample providing a control relative to the test sample of the subject; and (c) computing the differential expression of the miRNA from the subject, wherein the over expression of miR-1825 or the under expression of miR-484 is indicative of prostate cancer.

The subject to be screened for prostate cancer (also referred to herein as “test subject”) is a male mammal and preferably, a human male. A human subject can be of any race or ethnic group. As used herein, a “test sample” refers to any suitable biological sample including, but is not limited to, a tissue sample, whole tissues, blood, saliva, urine, cell culture, cell lysate and cell extract. In a preferred embodiment, the test sample is a urine sample.

As used herein a “normal sample” refers to a biological sample taken from a healthy control subject or a pool of healthy control subjects. A healthy control subject is a subject that does not suffer from prostate cancer.

Screening for prostate cancer is performed by assaying the miRNA expression level in the test sample for one or more of the following miRNAs:

miR-1825 (uccagugcccuccucucc - SEQ ID No: 1)
miR-484 (ucaggcucaguccccucccgau - SEQ ID No: 2)

and comparing the assayed miRNA expression level of the test sample to the miRNA expression level in a normal sample. The normal sample provides a control relative to the test sample of the subject being screened for prostate cancer.

miRNA expression levels can be assayed using methods well known in the art including, but not limited to, use of a polymerase chain reaction (PCR) assay, a microarray, or a Northern Blot. For example, a biological sample containing RNA is obtained from the test subject (“test sample”) and from a control subject or pool of control (“normal sample”). The test and normal samples are prepared using conventional methods to isolate the RNA. The presence and expression level of miR-1825 in the isolated RNA can be determined using a labelled probe specific for miR-1825 using conventional methods. Similarly, the presence and expression level of miR-484 in the isolated RNA can be determined using a labelled probe specific for miR-484. The Examples herein disclose the use of a microarray for detecting and quantifying expression levels for miR-1825 and miR-484 for test subjects and control subjects. It will be understood that miRNA expression levels can be assayed using any suitable method known in the art and it not limited to the use of microarrays.

Differential expression of miR-1825 and/or miR-484 is computed by analyzing the statistically significant association and/or a statistically significant correlation between an increase or decrease of miR-1825 and/or miR-484 expression levels in the test subject compared to the control subject (or pool of control subjects). Wherein an analysis identifies a statistical association (e.g.: a significant association) between an increase in miR-1825 expression level in the test subject, the over expression of miR-1825 is indicative of prostate cancer. Wherein an analysis that identifies a statistical association (e.g.: a significant association) between a decrease in miR-484 expression level in the test subject, the under expression of miR-484 is also indicative of prostate cancer. In one embodiment, the method for screening for prostate cancer can be performed by computing the differential expression of miR-1825 alone. In another embodiment, the method for screening for prostate cancer can be performed by computing the differential expression of miR-484 alone. In a further embodiment, the method for screening for prostate cancer can be performed by computing the differential expression of both miR-1825 and miR-484. A computing device having one or more processors configured to implement the step of computing the differential expression of the miRNA from the subject can be employed.

In a further embodiment, the method comprises providing a diagnosis of prostate cancer to the subject based on the differential expression of the miRNA. The diagnosis of prostate cancer is the affirmation of the presence of the disease based on the differential expression miR-1825, miR-484 or both.

In another embodiment, provided is a method for screening for benign prostatic hyperplasia in a subject comprising the steps of: (a) assaying the miRNA expression level in a test sample from the subject to be screened for benign prostatic hyperplasia; (b) comparing the assayed miRNA expression level of the subject to the miRNA expression level in a normal sample providing a control relative to the test sample of the subject; and (c) computing the differential expression of the miRNA from the subject, wherein the under expression of miR-498 is indicative of benign prostatic hyperplasia.

The subject to be screened for benign prostatic hyperplasia (also referred to herein as “test subject”) is a male mammal and preferably, a human male. A human subject can be of any race or ethnic group. As used herein, a “test sample” refers to any suitable biological sample including, but is not limited to, a tissue sample, whole tissues, blood, saliva, urine, cell culture, cell lysate and cell extract. In a preferred embodiment, the test sample is a urine sample.

As used herein a “normal sample” refers to a biological sample taken from a healthy control subject or a pool of healthy control subjects. A healthy control subject is a subject that does not suffer from benign prostatic hyperplasia.

Screening for benign prostatic hyperplasia is performed by assaying the miRNA expression level in the test sample for:

(SEQ ID No. 3)
miR-498 - uuucaagccagggggcguuuuuc

and comparing the assayed miRNA expression level of the test sample to the miRNA expression level in a normal sample. The normal sample provides a control relative to the test sample of the subject being screened for benign prostatic hyperplasia.

Methods for assaying the miRNA expression levels in the test sample and the normal sample can be performed using conventional methods as described above. The presence and expression level of miR-498 in isolated RNA obtained from the test sample and normal sample can be determined using a labelled probe specific for miR-498 using conventional methods. The Examples herein disclose the use of a microarray for detecting and quantifying expression levels for miR-498 for test subjects and control subjects. It will be understood that miR-498 expression levels can be assayed using any suitable method known in the art and is not limited to the use of microarrays.

Differential expression of the miR-498 expression levels is computed by analyzing the statistically significant association and/or a statistically significant correlation between the increase or decrease of miR-498 expression levels in the test subject and in the control subject (or pool of control subjects). Wherein an analysis that identifies a statistical association (eg: a significant association) between a decrease in miR-498 expression level in the test subject, the under expression of miR-498 is indicative of benign prostatic hyperplasia. A computing device having one or more processors configured to implement the step of computing the differential expression of the miRNA from the subject can be employed.

In a further embodiment, the method comprises providing a diagnosis of benign prostatic hyperplasia to the subject based on the differential expression of the miRNA. The diagnosis of benign prostatic hyperplasia is the affirmation of the presence of the disease based on the differential expression of miR-498.

While only specific embodiments of the invention have been described, it is apparent that variations can be made thereto without departing from the scope of the invention. The invention is further illustrated by the following examples, which are not to be construed in any way as imposing limitations upon the scope thereof. It is the intention in the appended claims to cover all variations that may fall within the true scope of the invention.

### EXAMPLE 1

### Deregulation of miR-1825 and miR-484 in Prostate Cancer

Urine samples were collected from 29 Egyptian males. The samples were acquired from among three groups of individuals, each described by a questionnaire completed by the patients' attending physician. Overall, urine samples were collected from eight individuals diagnosed with Prostate Cancer (PCa), twelve individuals with Benign Prostatic Hyperplasia (BPH) and from ten healthy males (Healthy Control). Urine samples were collected into 50 mL Corning tubes containing Norgen's Urine Preservative Solution (Cat#18126) (Norgen Biotek, Thorold, ON, Canada). Urine samples were collected mid-stream in order to minimize first flow contamination, Total RNA was isolated from 2.5 mL of each urine sample using the Urine Total RNA Purification Maxi Kit (Slurry Format, Cat#29600; Norgen Biotek, Thorold, ON, Canada). Purified RNA from each sample within a group was then pooled, resulting in three pooled samples: PCa, BPH and Healthy Control.

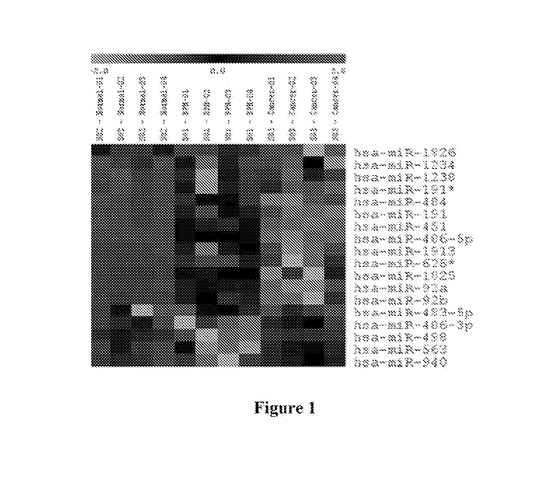

Differential expression analysis of miRNAs between the pooled samples was performed using LC Science's μParaflo™ MiRNA Microarray technology. To determine fold change, the signal intensity of each miRNA (in pooled PCa or BPH) was divided against the corresponding miRNA level for the Healthy Control group and the LOG2 of the quotient was then obtained. Of the 894 miRNAs assayed, eighteen were found to differ significantly in their levels between the three groups (ANOVA p-value <0.01) (FIG. 1). From these eighteen, only certain miRNAs were selected for further analysis. Using both a distinct pattern of pooled microRNA deregulation or literature suggesting that a miRNA's mRNA target was potentially involved in cancer (targets determined using TargetScan and MiRanda databases as outlined by Griffiths-Jones et al. in 2006), eight of miRNAs were selected for further expression analysis among individual urine samples. The selected miRNAs included miRs: 1234, 1238, 1913, 486-5p, 1825, 484, 483-5p and 498.

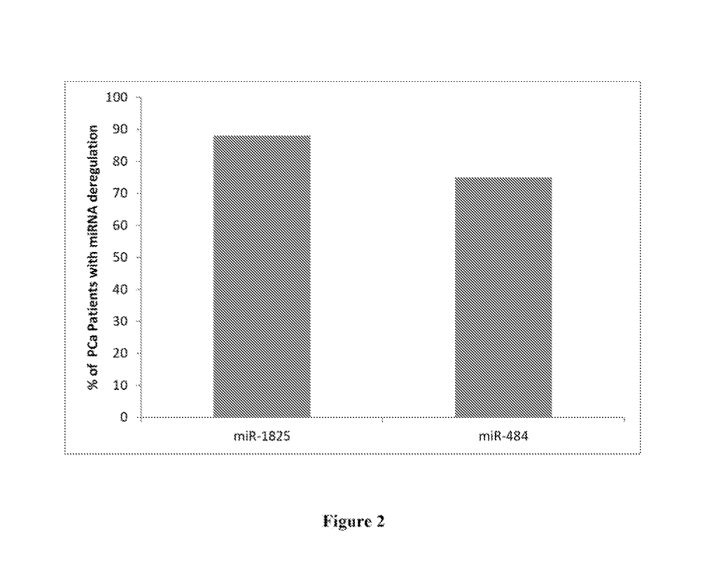

Seven of the selected miRNAs were, according to microarray data on pooled samples, deregulated in the PCa group compared to the healthy control group. In order to evaluate those miRNA's potential as candidate biomarkers for PCa, the relative expression of each was examined in all individual PCa samples by using RT-qPCR. To allow for a comparison of expression levels between samples, the expression of each miRNA was normalized against each sample's own content of 5S ribosomal RNA. Relative expression between each PCa sample and the average expression of the healthy control group was then calculated as LOG2 fold change values using the following equation: LOG2 (2-ΔCt (target miRNA) PCa/2-ΔCt (target miRNA) Healthy Control. For PCR data, an absolute LOG2 value of one or more was considered deregulated, while a value of less than one was considered minimally deregulated/unchanged. From this group, 2 miRNAs were identified that were deregulated in a substantial majority of the PCa samples: miR-1825 and miR-484. MiR-1825 was up-regulated in seven out of eight PCa samples (88%) and miR-484 was down-regulated in six out of the eight PCa samples (75%) (FIG. 2). Thus, in this study, miR-1825 was found to be increased and miR-484 was found to be decreased in prostate cancer patients compared to the Healthy Controls.

### EXAMPLE 2

### Deregulation of miR-498 in Benign Prostatic Hyperplasia

Urine samples were collected from 29 Egyptian males. The samples were acquired from among three groups of individuals, each described by a questionnaire completed by the patients' attending physician. Overall, urine samples were collected from eight individuals diagnosed with Prostate Cancer (PCa), twelve individuals with Benign Prostatic Hyperplasia (BPH) and from ten healthy males (Healthy Control). Urine samples were collected into 50 mL Corning tubes containing Norgen's Urine Preservative Solution (Cat#18126) (Norgen Biotek, Thorold, ON, Canada). Urine samples were collected mid-stream in order to minimize first flow contamination. Total RNA was isolated from 2.5 mL of each urine sample using the Urine Total RNA Purification Maxi Kit (Slurry Format, Cat#29600; Norgen Biotek, Thorold, ON, Canada). Purified RNA from each sample within a group was then pooled, resulting in three pooled samples: PCa, BPH and Healthy Control.

Differential expression analysis of miRNAs between the pooled samples was performed using LC Science's μParaflo™ MiRNA Microarray technology. To determine fold change, the signal intensity of each miRNA (in pooled PCa or BPH) was divided against the corresponding miRNA level for the healthy control group and the LOG2 of the quotient was then obtained. Of the 894 miRNAs assayed, eighteen were found to differ significantly in their levels between the three groups (ANOVA p-value <0.01) (FIG. 1). From these eighteen, only certain miRNAs were selected for further analysis. Using both a distinct pattern of pooled microRNA deregulation or literature suggesting that a miRNA's mRNA target was potentially involved in cancer (targets determined using TargetScan and MiRanda databases as outlined by Griffiths-Jones et al. in 2006), eight of miRNAs were selected for further expression analysis among individual urine samples. The selected miRNAs included miRs: 1234, 1238, 1913, 486-5p, 1825, 484, 483-5p and 498.

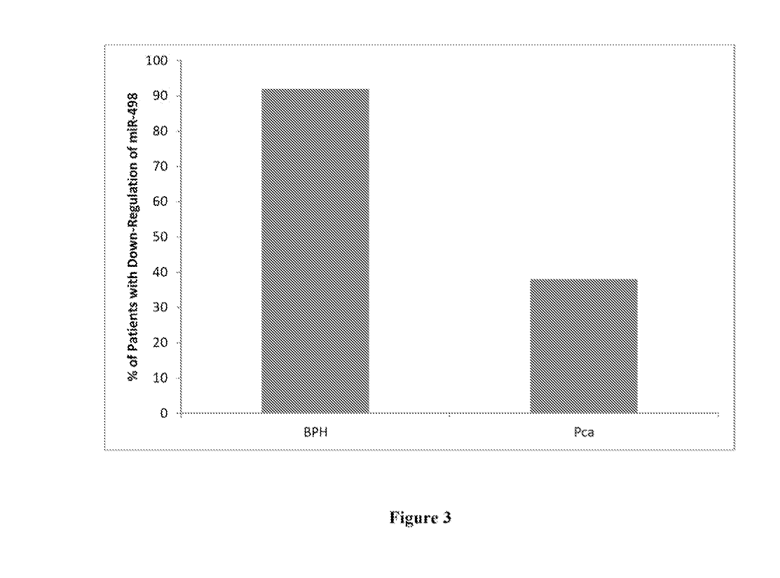

One of the selected miRNAs, miR-498 was observed by microarray to be down-regulated primarily in pooled BPH. In order to evaluate this miRNA's potential as a candidate biomarker for BPH, the relative expression was examined in all individual BPH and PCa samples by using RT-qPCR. To allow for a comparison of expression levels between samples, the expression of miR-498 was normalized against each sample's own content of 5S ribosomal RNA. Relative expression between each BPH or PCa sample and the average expression of the healthy control group was then calculated as described in Example 1. It was found that MiR-498 was down-regulated in eleven of the initially collected twelve BPH samples (92%), and that miR-498 was not deregulated in a significant number of PCa samples (FIG. 3). Thus, in this study, miR-498 was found to be decreased in BPH patients but not in PCa patients or healthy controls. Therefore, a decrease in miR-498 can be used to diagnose BPH and help to prevent unnecessary biopsies in individuals who present with enlarged prostates.

While the invention has been described with reference to preferred and example embodiments, it will be understood by those skilled in the art that a variety of modifications, additions and deletions are within the scope of the invention, as defined by the following claims.

1. A method for screening for prostate cancer in a subject comprising the steps of:
a. assaying the miRNA expression level in a test sample from the subject to be screened for prostate cancer;
b. comparing the assayed miRNA expression level of the subject to the miRNA expression level in a normal sample providing a control relative to the test sample of the subject; and
c. computing the differential expression of the miRNA from the subject, wherein the over-expression of miR-1825 or the under expression of miR-484 is indicative of prostate cancer.

2. The method according to claim 1, wherein the method comprises providing a diagnosis of prostate cancer to the subject based on the differential expression of the miRNA.

3. The method according to claim 1, wherein the biological sample is urine.

4. The method according to claim 1, wherein the subject is human.

5. The method according to claim 1, wherein the assaying is conducted using a polymerase chain reaction (PCR) assay, a microarray or a Northern blot.

6. A method for screening for benign prostatic hyperplasia in a subject comprising the steps of:
a. assaying the miRNA expression level in a test sample from the subject to be screened for benign prostatic hyperplasia;
b. comparing the assayed miRNA expression level of the subject to the miRNA expression level in a normal sample providing a control relative to the test sample of the subject; and
c. computing the differential expression of the miRNA from the subject, wherein the under expression of miR-498 is indicative of benign prostatic hyperplasia.

7. The method according to claim 6, wherein the method comprises providing a diagnosis of benign prostatic hyperplasia to the subject based on the differential expression of the miRNA.

8. The method according to claim 6, wherein the biological sample is urine.

9. The method according to claim 6, wherein the subject is human.

10. The method according to claim 6, wherein the assaying is conducted using a polymerase chain reaction (PCR) assay, a microarray, or a Northern Blot.


SEQUENCE LISTING (3 sequences)

SEQ ID NO: 1 - RNA, 18 nt, Homo sapiens
uccagugcccuccucucc

SEQ ID NO: 2 - RNA, 22 nt, Homo sapiens
ucaggcucaguccccucccgau

SEQ ID NO: 3 - RNA, 23 nt, Homo sapiens
uuucaagccagggggcguuuuuc